(19) United States
(12) Patent Application Publication
(10) Pub. No.: US 2019/0045735 A1
(43) Pub. Date: Feb. 14, 2019

(54) HIGH ALPHA LINOLENIC ACID FLAX

(71) Applicants: Nathan GOLAS, Fisher Branch (CA); Lillian PETERSON, Fisher Branch (CA); Arvind KUMAR, Fisher Branch (CA)

(72) Inventors: Nathan GOLAS, Fisher Branch (CA); Lillian PETERSON, Fisher Branch (CA); Arvind KUMAR, Fisher Branch (CA)

(21) Appl. No.: 16/079,653

(22) PCT Filed: Feb. 27, 2017
(86) PCT No.: PCT/US17/19680

§ 371 Date: Aug. 24, 2018

Related U.S. Application Data
(60) Provisional application No. 62/300,364, filed on Feb. 26, 2016.

(51) Int. Cl. A01H 6/58; A01H 5/10; C07C 57/03

(52) U.S. Cl. CPC A01H 6/58; C07C 57/03; A01H 5/10

(57) ABSTRACT

The present invention is directed to simple a cultivar of the flax plant (Linum usitatissimum) which produces a novel profile of linolenic acid. The plant, the oil products and the unique genes of the cultivar are described. A cultivar producing a seed with high concentration of alpha linolenic acid is further described by genome profile including cDNA and simple sequence repeat (SSR or microsatellite) regions. The cultivar can also be identified by its novel flaxseed oil profile.

### CROSS-REFERENCE TO PRIOR FILED APPLICATION

This application claims priority to an earlier filed U.S. provisional application Ser. No. 61/300,364 filed on Feb. 26, 2016, which is herein incorporated by reference in its entirety.

### FIELD OF THE INVENTION

This disclosure generally relates to a flax plant cultivar which produces a novel profile of linolenic acid. The plant, the oil products and the unique genes of the cultivar are described. A cultivar producing a seed with a high concentration of alpha linolenic acid is further described by genome profile. Chemical analysis of flaxseed oil, Genomic SSR, cDNA and protein sequencing are used to describe the cultivar.

### BACKGROUND

Flax is an annual, self-pollinating plant of the family Linaceae with an ancient history of use by humans. Flax varieties may be grown for fiber from the stems or for oil from seeds. Fiber flax, such as Hermes, is almost unbranched whereas oilseed flax such as Normandy, Bethune, or Sorrell are highly branched or bushy. Some efforts are ongoing to combine characteristics of oilseed flax and fiber flax. Flax oil is a natural source of essential fatty acids alpha linolenic acid (ALA) and linoleic acid (LA). The fatty acid profile of oil from fiber or oilseed flax is characteristic of each variety. For example, Solin has an extremely low alpha linolenic acid content and higher linoleic acid content. This variety was intended as a replacement for other cooking oils. Wild type Normandy, Bethune and Sorrell have higher alpha linolenic acid content i.e. 48 to 60% and lower linoleic acid content i.e. 16% as compared to Solin (FIG. 1). High alpha linolenic acid flax has an extremely high alpha linolenic acid content (68% or greater) and a lower linoleic acid content (10%) than other flax varieties or cultivars (FIG. 1). Characteristics of high alpha linolenic acid flax oil are more completely described in WO 2007/051302 and FDA GRN #256 both included herein by reference.

Alpha linolenic acid is also known as plant source omega 3 (C₁₈:3n3). Linoleic acid is also known as omega 6 fatty acid or (C₁₈2n6. Alpha linolenic acid and linoleic acid are known as essential fatty acids because the human body cannot endogenously produce these fats. The individual must consume these fatty acids in the diet. The body uses these fatty acids in numerous ways which have implications for general health including improvements in cardiovascular function, brain and eye development, skin health, etc. The FDA has approved high alpha linolenic acid flax oil as Generally Recognized as Safe (GRAS). High alpha linolenic acid flax oil also benefits animal health and production. An increased dietary ALA intake reduces pregnancy losses for cattle, improves coat appearance and health far horses and dogs, reduces weaning time for pigs and increases the resistance of animals to disease. High alpha linolenic acid flax/also has implications for industrial uses. After epoxidation, a high alpha linolenic acid content results in an epoxidized natural oil with a higher than average oxirane value. Epoxies made with epoxidized high alpha linolenic acid are faster drying and farm stronger, more chemically resistant bonds. Similarly, alkyd resins based on high alpha linolenic acid flax oil are more resistant to solvents, stronger and more durable. Furthermore, such high alpha linolenic acid flax oil epoxies and alkyd resins are based on ‘green’ chemistry and as such can be used to replace older technologies and benefit the environment. Therefore, high alpha linolenic acid flax oil has economic importance with applications in areas as diverse as human health, animal feed and industrial oils.

Alpha linolenic acid content of oils from the different varieties and cultivars of flax is to a small part determined by environmental factors, A longer photoperiod growing season and cooler temperatures will result in an oil with higher alpha linolenic acid content for any particular variety/cultivar. For the most part, however, alpha linolenic acid content is determined genetically. Specifically, the alpha linolenic acid content of mature seeds is determined by the FAD3a and FAD3b genes. These genes encode omega 3/delta 15 desaturase enzymes capable of catalyzing a double bond in linoleic add to produce alpha linolenic acid (FIG. 5). Solin, a variety of flax with extremely low alpha linolenic acid content has mutations in the FAD3a and FAD3b genes as compared to wild type Normandy FAD genes. These mutations result in a truncated amino acid sequence which produced inactive FAD desaturase proteins and subsequently low levels of alpha linolenic acid.

The flax species has a high degree of variability. It has been bred to produce a wide range of cultivars each with various desirable characteristics. Genetic analysis of the flax genome reveals that the species is genetically suite to the production of cultivars. As much as 20% of the genome is composed of transposable elements. It is well suited to producing diverse cultivars of a variety of argobotanic characteristics. Therefore, a method of genetic description of the cultivars has been researched and developed.

The flax genome, in its entirety, may be characterized by patterns of simple sequence repeat regions, among other methods. A simple sequence repeat (SSR) region (also known as microsatellite or variable length tandem repeat region) is a DNA sequence which consists of repeated nucleotide units. The total length of this particular region is variable, depending on the length of the nucleotide unit sequence itself and on how many times the nucleotide unit is repeated. SSR regions are found at many loci in the genome. Each SSR region may consist of different DNA repeated units and may be of different lengths, Each locus is identified by a unique primer sequence. Each locus is polymorphic and may have many alleles i.e. the SSR region at any one particular locus varies between individuals. This polymorphism results in a pattern of genomic SSR regions which is characteristic for a particular individual. The characteristic and unique pattern of SSR regions is the basis of genetic markers, DNA fingerprinting, paternity testing, individual identification, quantitative trait loci mapping, genetic diversity studies, association mapping and fingerprinting cultivars. In plants, the characteristic SSR regions are used for cultivar identification and evaluation of DNA variation. In flax, twenty-eight SSR markers have been reported. A large-scale study of SSR markers in flax identified the lineage of many varieties of flax. Based upon SSR data high alpha linolenic acid flax forms a new accession.

It is recently understood high alpha linolenic acid flax is useful and desirable in variety of nutritional and industrial uses, It is well-known alpha linolenic acid is an essential oil for human and other mammals. It is also understood high alpha linolenic acid flax (linseed) oil containing 65% or greater alpha linolenic acid can be used in the production of alkyd resins epoxidized oils, coatings, paints, enamels, varnishes, anti-spalling surface concrete preservatives, solidified linseed oils, maleinated linseed oils, epoxies, inks, zein film coatings and other useful applications recognizable to one skilled in the art. The products of high alpha linolenic acid are often stronger and dry faster compared to similar products made with wildtype linolenic flax (linseed) oil. The industrial and agricultural uses of high alpha linolenic acid are well described in U.S. Pat. Bo. 9,179,660 to Peterson and Gola.

It is understood that reduction of linolenic acid to alpha linolenic acid in flax is regulated by the FAB3A and FAB3B genes. Each of which codes of an omega 3/delta15 desaturase. The omega 3/delta14 desaturase is capable of catalyzing the formation of a double bond in linoleic acid forming alpha linolenic acid. Modification of the FAB3A/B gene in nucleic acid sequence and/or gene regulation is thought to control the production of alpha linolenic acid and modulate the ratio of alpha linolenic acid to linoleic acid.

There exists a need for genetic characterization of biologic profile of a flax plant capable of producing high alpha linolenic acid flax seed. Such a plant is desirable for the production of high alpha linolenic acid flaxseed oil, particular of flaxseed oil with 18:3 linolenic acid at or above 70%, more preferably above 75%. It is also desirable for the cultivar to produce a high alpha linolenic acid seed than also comprise linoleic acid and oleic acid. Such a plant is also desirable as a parent for the production of cultivars with these desirable characteristics and other phenotypes.

### SUMMARY OF INVENTION

Characteristics of high alpha linolenic acid flax such as SSR regions and FAD3a/b gene sequences are unique. One embodiment of the invention is a set of chromosomes of a high alpha linolenic acid flax plant which can be characterized by genome comprising a pattern of simple sequence repeats which has 85%, 87.5 90%, 91%, 92%, 93%, 94%, 95%, 96%, 97%, 98% or 99% equality to base pair length of the simple sequence repeats patterns of cultivar M6552 at the locus defined by primer pairs of SEQ ID NO 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34; and wherein the seeds of the plant possess an alpha linolenic acid content of greater than 65%, 70%, 71%, 72%, 73%, 74% or 75% (weight % of the cold pressed oil). A preferred embodiment of the invention is like the above yet, wherein the simple sequence repeats pattern defined by primer pair SEQ ID NO: 19 and SEQ ID NO: 20 is about equal to 226 base pairs and at least one of the following is also true: the simple sequence repeats pattern defined by primer pair SEQ ID NO: 1 and SEQ ID NO: 2 is equal to or greater than about 231 base pairs; the simple sequence repeats pattern defined by primer pair SEQ ID NO: 2 and SEQ ID NO: 4 is equal to or greater than about 197 base pairs; and, the simple sequence repeats pattern defined by primer, pair SEQ ID NO: 9 and SEQ ID NO: 10 is equal to about 371 base pairs. Another preferred embodiment is like the first embodiment yet wherein the simple sequence repeats pattern defined by primer pair SEQ ID NO: 13 and SEQ ID NO: 14 is greater than about 305 base pairs.

Another embodiment of the invention is a nucleotide sequence comprising a nucleic acid sequence with 65%, 70%, 75%, 80%, 85%, 87.5%, 90%, 91%, 92%, 93%, 94%, 95%, 96%, 97%, 98% or 99% identity to the coding sequence of the FAD3gene listed in SEQ ID NO: 35; wherein the sequence encodes a protein with fatty acid desaturase activity sufficient for use in the synthesis of alpha linolenic acid from linoleic acid.

Another embodiment of the invention is a nucleotide sequence comprising a nucleic acid sequence with 65%, 70%, 75%, 80%, 85%, 87.5%, 90%, 91%, 92%, 93%, 94%, 95%, 96%, 97%, 98% or 99% identity to the coding sequence of the FAD3b protein as listed in SEQ ID NO: 41, wherein the sequence encodes a protein with fatty acid desaturase activity sufficient for use in the synthesis of alpha linolenic acid.

Another embodiment of the invention is a protein comprising an amino acid sequence with 65%, 70%, 75%, 80%, 85%, 87.5%, 90%, 91%, 92%, 93%, 94%,95%, 96%, 97%, 98% or 99% identity to the amino acid sequence of the FAD3a protein as listed in SEQ ID NO: 36, wherein the protein is capable of catalyzing the formation of a double bond in linoleic acid to produce alpha linolenic acid.

Another embodiment of the invention is a protein comprising an amino acid sequence with 65%, 70%, 75%, 80%, 85%, 87.5%, 90%, 91%, 92%, 93%, 94%, 97%, 98%, or 99% identity to the amino acid sequence of the FAD3b protein as listed in SEQ ID SEQ ID NO: 42, wherein the protein is capable of catalyzing the formation of a double bond in linoleic acid to produce alpha linolenic acid.

Another embodiment of the invention is a cDNA sequence derived from high alpha linolenic acid flax wherein, the cDNA sequence has at least about at least one of the mutations depicted by the cDNA of the FAD3a of high alpha linolenic acid flax as listed in SEQ ID NO: 35. This embodiment of the invention is more preferably a cell, particular a plant cell, yeast cell, or bacteria cell, that has been transformed with the cDNA. Most preferably, a multi-cellular organism comprising a cell with the cDNA as listed in SEQ ID NO: 35.

Another embodiment of the invention is a cDNA sequence derived from high alpha linolenic acid flax wherein, the cDNA sequence has at least about at least one of the mutations depicted by the cDNA of the FAD3b of high alpha linolenic acid flax as listed in SEQ ID NO: 41. This embodiment of the invention is more preferably a cell, particular a plant cell, yeast cell, or bacteria cell, that has been transformed with the cDNA. Most preferably, a multi-cellular organism comprising a cell with the cDNA as listed in SEQ ID NO: 41.

Another embodiment of the invention is a FAD3a protein comprising at least one of the mutations, shown in FIG. 8, of NoFad3A.pro when compared to BeFad3A.pro or NmFad3A.pro. More preferably in this embodiment, the FAD3a protein is further capable of catalyzing the formation of a double bond in a linolenic fatty acid. Even more preferable this protein is contained with a cell and, most preferably the embodiment is an organism that comprising the cell containing the protein.

Another embodiment of the invention is a FAD3b protein comprising at least one of the mutations, shown in FIG. 9, of NoFad3B.pro when compared to BeFad3B.pro or NmFad3B.pro. More preferably in this embodiment, the FAD3a protein is further capable of catalyzing the formation of a double bond in a linolenic fatty acid. Even more preferable this protein is contained with a cell and, most preferably the embodiment is an organism that comprising the cell containing the protein.

Another embodiment of the invention is a Linum usitatissium plant comprising the LU17 SSR of 308 bp and wherein the percentage of alpha linolenic acid compared to total oil is greater than about 70.1% More preferably, the Linum usitatissium plant further comprising the whole pattern of SSRs depicted in FIG. 3, column “High Alpha”.

Another embodiment of the invention is a high alpha linolenic acid flax plant having the modified genes as listed in SEQ ID NO: 35 and SEQ ID NO: 41 and expressing the amino acid sequence as listed in SEQ ID NO: 36 and SEQ ID NO: 42. ln another embodiment, the invention features an isolated nucleic acid sequence which encodes the FAD3A gene in high alpha linolenic acid flax.

In another embodiment, the invention features an isolated nucleic acid sequence which encodes the FAD3B gene in high alpha linolenic acid flax. In a further embodiment of the invention, the FAD3A and FAD3B genes encode amino acid sequences unique to high alpha linolenic acid flax. The protein formed from this amino acid sequence are desaturases i.e. catalyze the formation of double bands. Specifically, these proteins desaturase linoleic acid to form alpha linolenic acid.

Another embodiment of the invention is a cultivar of the flax plant, wherein the flaxseed comprises more than 60%, 65%, 70% or 73% alpha linolenic acid, more that 5%, 6%, 7%, 8%, 9% or 10% linoleic acid and more than 5%, 6%, 7%, 8% 9% or 10% oleic acid (weight percentages of the cold pressed oil).

### BRIEF DESCRIPTION OF THE FIGURES

For a more complete understanding of the disclosure, reference should be made to the following detailed description and accompanying drawing figures wherein:

FIG. 1. Typical total oil content and omega 3 alpha linolenic acid levels for different cultivars of flax (Linum usitatissimum).

FIG. 2. Primer sequences for each simple sequence repeat loci tested in high alpha linolenic acid flax. SEQ ID NOs 1-34 are shown.

FIG. 3. Length in base pairs (bp) of SSR regions for alleles at each locus 1 O identified in various varieties of flax including high alpha linolenic flax (M6552),extremely low linolenic flax (Linola), intermediate linolenic flax (Shubhara), conventional oilseed flax (Bethune, Normandy, Sorrell) and a fiber flax (Hermes).

FIG. 4. Comparison of SSR regions of M6552 Norcan to other varieties of flax with varying alpha linolenic acid content. Length (bp) of SSR regions for alleles at each locus identified in various varieties of flax including high alpha linolenic flax (M6552), extremely low linolenic flax (Unola), intermediate linolenic flax (Shubhara), conventional wild-type oilseed flax (Bethune, Normandy, Sorrell) and a fiber flax (Hermes).

FIG. 5. Alpha linolenic acid and other fatty acid synthesis in plants.

FIG. 6. FAD3A gene nucleotide sequence alignment among high alpha linolenic acid flax M6552 (NoFad3A.seq) (SEQ ID NO: 35), wild type Bethune (BeFad3A.seq) (SEQ ID NO: 37) and wild type Normandy (NmFad3A.seq) (SEQ ID NO: 39).

FIG. 7. FAD3A amino acid sequence alignment among high alpha linolenic acid flax M6552 (NoFad3A.pro) (SEQ IS NO: 36), wild-type Bethune (BeFad3A.pro) (SEQ ID NO: 38) and wild-type Normandy (Nm Fad3A.pro) (SEQ ID NO: 40).

FIG. 8. FAD3B gene nucleotide sequence alignment among high alpha linolenic acid flax M6552 (NoFad38 .seq) (SEQ ID NO: 41), wild type Bethune (BeFad3B.seq) (SEQ ID NO: 43) and wild type Normandy (NmFad38.seq) (SEQ ID NO: 45).

FIG. 9. FAD3B amino acid sequence alignment among high alpha linolenic acid flax M6552(No Fad3B.pro) (SEQ ID NO: 42), wild-type Bethune (BeFad3A.pro) (SEQ ID NO: 44) and wild-type Normandy (NmFad3B.pro) (SEQ ID NO: 46).

FIG. 10. A table comparing the M6552 flax cultivar to conventional flaxseed oil

FIG. 11. A table comparing the M6552 flax cultivar to other vegetable oils such as canola, corn, olive, peanut, safflower, soybean, sunflower and walnut oils

FIG. 12 A table showing the molecular formulas for the major fatty acid components of the M6552 Cultivar including alpha linolenic acid, linoleic, oleic, stearic and palmitic acid.

While specific embodiments are illustrated in the figures, with the understanding that the disclosure is intended to be illustrative, these embodiments are not intended to limit the invention described and illustrated herein.

### DETAILED DESCRIPTION

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. Throughout the specification, the seeds and oils are described by percentages of certain compounds, these percentages are based on the weight percent of the cold pressed oil or the cold pressed oil from the seed,

### Definitions

The terms “high linolenic acid linseed oil” and “high linolenic acid flax seed oil” and “High alpha linolenic acid flax (linseed) oil” are used interchangeably herein and refer to oil, for example unmodified or natural oil, that is, oil that following extraction from flax seeds has not been chemically, enzymatically or otherwise modified to increase the alpha linolenic content thereof, derived from flax seed having at least 65% alpha linolenic acid of total fatty acids, or 65-95% alpha linolenic acid, 65-94% alpha linolenic acid, 65-93% alpha linolenic acid, 65-92% alpha linolenic acid, 65-91% alpha linolenic acid, 65-90% alpha linolenic acid, 65-89% alpha linolenic acid, 65-88% alpha linolenic acid, 65-87% alpha linolenic acid, 65-86% alpha linolenic acid, 65-85% alpha linolenic acid, 65-84% alpha linolenic acid, 65-83% alpha linolenic acid, 65-82% alpha linolenic acid, 65-81% alpha linolenic acid, 65-80% alpha linolenic acid, 65-79% alpha linolenic acid, 65-78% alpha linolenic acid, 65-77% alpha linolenic add, 65-76% alpha linolenic acid, 65-75% alpha linolenic acid, 65-74% alpha linolenic acid, 65-73% alpha linolenic acid, 65-72% alpha linolenic acid, 65-71% alpha linolenic acid, 65-70% alpha linolenic acid, 65-69% alpha linolenic acid, 65-68% alpha linolenic acid, 65-67% alpha linolenic acid, 65-66% alpha linolenic acid, 67-95% alpha linolenic acid, 67-94% alpha linolenic acid, 67-93% alpha linolenic acid, 67-92% alpha linolenic acid, 67-91% alpha linolenic acid, 67-90% alpha linolenic acid, 67-89% alpha linolenic acid, 67-88% alpha linolenic acid, 67-87% alpha linolenic acid, 67-86% alpha linolenic acid, 67-85% alpha linolenic acid, 67-84% alpha linolenic acid, 67-83% alpha linolenic acid, 67-82% alpha linolenic acid, 67-81% alpha linolenic acid, 67-80% alpha linolenic acid, 67-79% alpha linolenic acid, 67-78% alpha linolenic acid, 67-77% alpha linolenic acid, 67-76% alpha linolenic acid, 67-75% alpha linolenic acid, 67-74% alpha linolenic acid, 67-73% alpha linolenic acid, 67-72% alpha linolenic acid, 67-71% alpha linolenic acid, 67-70% alpha linolenic acid, 67-69% alpha linolenic acid, 67-68% alpha linolenic acid, 70-95% alpha linolenic acid, 70-94% alpha linolenic acid, 70-93% alpha linolenic acid, 70-92% alpha linolenic acid, 70-91% alpha linolenic acid, 70-90% alpha linolenic acid, 70-89% alpha linolenic acid, 70-88% alpha linolenic acid, 70-87% alpha linolenic acid, 70-86% alpha linolenic acid, 70-85% alpha linolenic acid, 70-84% alpha linolenic acid, 70-83% alpha linolenic acid, 70-82% alpha linolenic acid, 70-81% alpha linolenic acid, 70-80% alpha linolenic acid, 70-79% alpha linolenic acid, 70-78% alpha linolenic acid, 70-77% alpha linolenic acid, 70-76% alpha linolenic acid, 70-75% alpha linolenic acid, 70-74% alpha linolenic acid, 70-73% alpha linolenic acid, 70-72% alpha linolenic acid, or 70-71% alpha linolenic acid.

High alpha linolenic acid flax (linseed) oil with greater than 65% alpha linolenic acid as described herein is produced by cold pressing High alpha linolenic acid flaxseed without the use of solvents or hexanes. This all natural process crushes the High alpha linolenic acid flax (linseed) seed to produce the oil. The High alpha linolenic acid flax (linseed) oil naturally contains a high alpha linolenic acid content as described herein. High alpha linolenic acid flaxseed with alpha linolenic acid content of greater than 65% is the result of careful plant breeding and in field selection as described in U.S. Pat. No. 6,870,077 and PCT Application WO03/064576 and included herein as reference. As will be appreciated by one of skill in the art, the varieties described in U.S. Pat. No. 6,870,077 and PCT Application WO03/064576 may be bred with other flax varieties to generate novel High alpha linolenic acid varieties with other desirable traits as described therein.

The terms “low linolenic flax (linseed) oil” and “regular flax (linseed) oil” and ‘normal flax (linseed) oil’ and “non-high linolenic (linseed) oil” are used interchangeably herein and refer to oil derived from flax seed having less than 65% alpha linolenic acid.

The terms “flaxseed oil” and “linseed oil” are used interchangeably herein, each refers to the same oil obtained from seeds of the flax plant (Linum usitatissimum).

As used herein, the term “conjugated double bonds” is art recognized and includes conjugated fatty acids (CFAs) containing conjugated double bonds. For example, conjugated double bonds include two double bonds in the relative positions indicated by the formula —CH.dbd.CH—CH.dbd.CH—. Conjugated double bonds form additive compounds by saturation of the 1 and 4 carbons, so that a double bond is produced between the 2 and 3 carbons.

As used herein, the term “fatty acids” is art recognized and includes a long-chain hydrocarbon based carboxylic acid. Fatty acids are components of many lipids including glycerides and which may be saturated or unsaturated. “Unsaturated” fatty acids contain cis double bonds between the carbon atoms. “Polyunsaturated” fatty acids contain more than one double bond and the double bonds are arranged in a methylene interrupted system (—CH.dbd.CH—CH.sub.2-CH.dbd.CH.

Fatty acids are described herein by a numbering system in which the number before the colon indicates the number of carbon atoms in the fatty acid, whereas the number after the colon is the number of double bonds that are present. In the case of unsaturated fatty acids, this is followed by a number in parentheses that indicates the position of the double bonds. Each number in parenthesis is the lower numbered carbon atom of the two connected by the double bond. For example, linoleic acid can be described as 18:2(9, 12) indicating 18 carbons, one double bond at carbon 9 and 18 carbons, two double bonds at carbons 9 and 12, respectively; and oleic acid can be described as 18:1(9).

As used herein, the term “conjugated fatty acids” is art recognized and includes fatty acids containing at least one set of conjugated double bonds, The process of producing conjugated fatty acids is art recognized and includes, for example, a process similar to desaturation, which can result in the introduction of one additional double bond in the existing fatty acid substrate.

As used herein, the term “linoleic acid” is art recognized and includes an 18 carbon polyunsaturated fatty acid molecule (C₁₇H₂₉COOH) which contains 2 double bonds (18:2(9, 12)), The term “Conjugated linoleic acid” (CLA) is a general term for a set of positional and geometric isomers of linoleic acid that possess conjugated double bonds, in the cis or trans configuration.

As used herein, the term “desaturase” is art recognized and includes enzymes that are responsible for introducing conjugated double bonds into acyl chains. In the present invention, for example, the .omega.-3 desaturase/delta15 desaturase from Linum usitatissimum is a desaturase that can introduce a double bond at position 15 of linoleic acid.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85%, 90% or 95% identical to each other remain hybridized to each other. Such stringent conditions are known to those skilled in the art and can be found, inter alia, in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4×sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15 M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SOS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

As used herein “percent identity” is a mathematical comparison of the relatedness of two sequences of nucleic acids or two sequences of amino acids, including longer sequences of amino acids that may be referred to as polypeptides or proteins. The comparison of sequences and determination of percent identity between two sequences can be accomplished using a mathematical algorithm. One skilled in the art will recognize there are several accepted methods of determining percent identity. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. A preferred, non-limiting example of parameters to be used in conjunction with the GAP program include a Blosum 62 scoring matrix with a gap penalty of 12, a gap extend penalty of 4, and a frameshift gap penalty of 5. In another embodiment, the percent identity between two amino acid or nucleotide sequences is determined using the algorithm of E. Meyers and W. Miller (Comput. Appl.. Blosci., 4:11-17 (1988)) which has been incorporated into the ALIGN program (version 2.0 or version 2.0U), using a PAM120 weight residue table, a gap length penalty of 12 and a gap penalty of 4.

It is well understood by one skilled in the art that many levels of sequence identity are useful in identifying polypeptides from other species or modified naturally or synthetically wherein such polypeptides have the same or similar function or activity. Useful examples of percent identities include, but are not limited to, 50%, 55%, 60%, 65%, 70%, 75%, 80%, 85%, 90% or 95%, or any integer percentage from 50% to 100%. Indeed, any integer amino acid identity from 50% to 100% may be useful in describing the present invention, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

The term “genome” as it applies to a plant cell encompasses not only chromosomal DNA found within the nucleus, but organelle DNA found within subcellular components (e.g., mitochondria, or plastid) of the cell.

As used herein, “codon-modified gene” or “codon-preferred gene” or “codon-optimized gene” is a gene having its frequency of codon usage designed to mimic the frequency of preferred codon usage of the host cell.

An “allele” is one of several alternative forms of a gene occupying a given locus on a chromosome. When all the alleles present at a given locus on a chromosome are the same, that plant is homozygous at that locus. If the alleles present at a given locus on a chromosome differ, that plant is heterozygous at that locus.

A “transgene” is a gene that has been introduced into the genome by a transformation procedure. A transgene can, for example, encode one or more proteins or RNA that is not translated into protein. However, a transgene of the invention need not encode a protein and/or non-translated RNA. In certain embodiments of the invention, the transgene comprises one or more chimeric genes, including chimeric genes comprising, for example, a gene of interest, phenotypic marker, a selectable marker, and a DNA for gene silencing.

As used herein, the term “locus” refers to a position on the genome that corresponds to a measurable characteristic (e.g., a trait). An SNP locus is defined by a probe that hybridizes to DNA contained within the locus.

As used herein, the term “marker” refers to a gene or nucleotide sequence that can be used to identify plants having a particular allele. A marker may be described as a variation at a given genomic locus. A genetic marker may be a short DNA sequence, such as a sequence surrounding a single base-pair change (single nucleotide polymorphism, or “SNP”). In a preferred use, the term “marker” refers to a profile of SSR at a particular locus or loci that characterize a particular allele,

Polymorphism: variation of the genetic sequence among alleles. An example is single nucleotide polymorphism where the gene sequence between alleles is changed by only one nucleotide.

As used herein, the term “SSR” refers to Simple Sequence Repeats or microsatellite. A region of the gene sequence which consists of repeated nucleotides or repeated units of a particular gene sequence Short Simple Sequence stretches occur as highly repetitive elements in all eukaryotic genomes. Simple sequence loci usually show extensive length polymorphisms. These simple sequence length polymorphisms (SSLP) can be detected by polymerase chain reaction (PCR) analysis and be used for identity testing, population studies, linkage analysis and genome mapping. A particular locus at which a SSR is found is identified by a primer sequence of DNA. The length of the SSR at each particular locus is characteristic and specific and can be used to identify cultivars in Linum usitatissimum. As used herein, the terms “satellite”, “minisatellite”, “microsatellite”, “short tandem repeat”, “STP”, “variable number of tandem repeats”, “VNTP” and “simple sequence repeat” are all considered to be synonymous with SSR.

Cultivar: A cultivated variety of a plant that has been deliberately selected for specific desirable characteristics such as the color of the flower, disease resistance, yield of crop etc., For the purpose of this patent, the gene sequences described herein are characteristic and unique to a cultivar of Linum usitatissimum which has been cultivated deliberately for high alpha linolenic acid content in the oil of the mature seed.

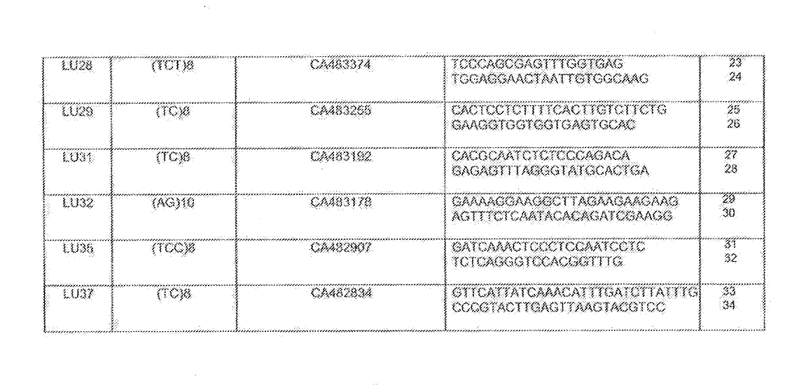

Primer: For the purposes of this patent, the primers are short strands of DNA which was hybridized to the target DNA at each of seventeen different loci. A list of the primers used herein is included in FIG. 2.

Locus (loci plural): For the purposes of this patent a locus is the specific DNA sequence on a chromosome at which a SSR region is located. Hyper variable: SSR or microsatellite regions are hyper variable in that the total number of repeated units may vary i.e. the total length of the SSR region may vary.

Some authors distinguish between the terms linseed and flaxseed, others do not. For some linseed may indicate flax used for oil, human food, livestock and pet food whereas the term flaxseed indicates flax used for fiber. However, others refer to linseed as flax used for industrial purposes, paints, epoxies, adhesives and flaxseed as flax used for human food, livestock and pet food. For the purposes of this patent the terms linseed and flaxseed are used interchangeably and are used to described flax used for any purpose i.e. used for oil, human food, pet food, fiber, industrial oil, paints epoxies, adhesives etc. Flax plant

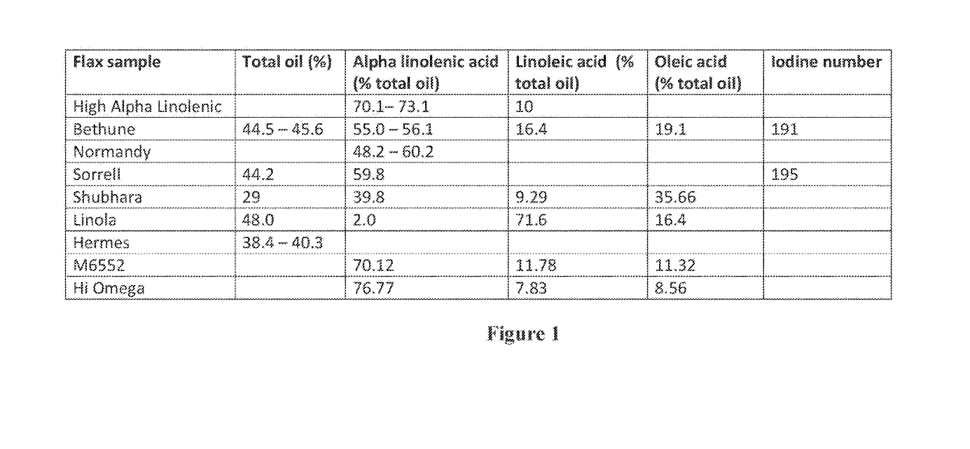

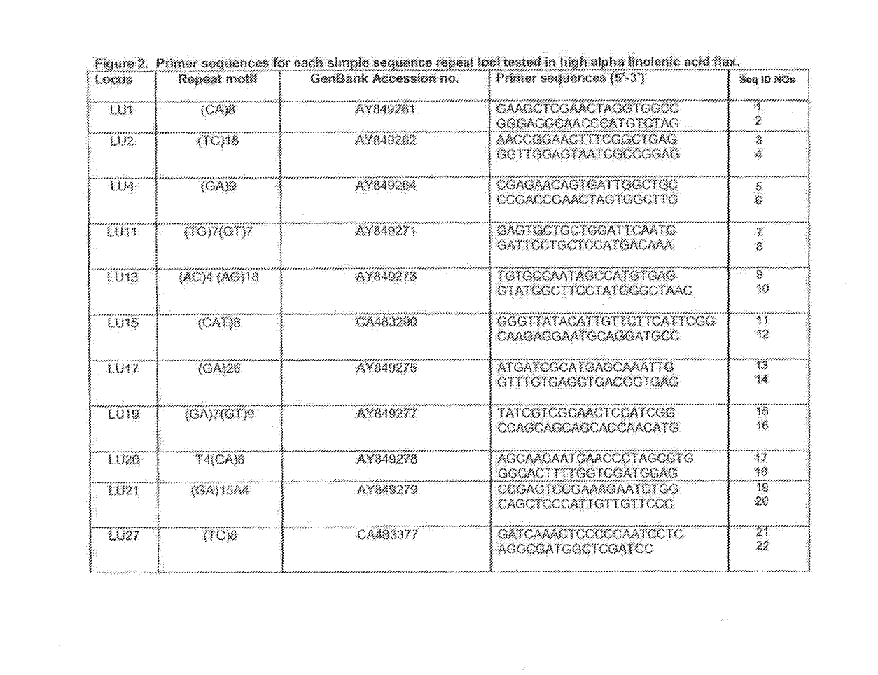

Flax is a self-pollinated diploid species with a chromosome number of 2n=30. Flax cultivars are homozygous. The flax genome of fiber flax has been completely characterized. High alpha linolenic acid flax was developed using conventional plant breeding methods. Such methods involve successive generations of inbreeding and are well known to those skilled in the art. High alpha linolenic acid flax is defined as flax cultivars which produce seeds containing oil with 65%, 70%, 75% or greater alpha linolenic acid. FIG. 1 provides the fatty acid profile and characteristics of high alpha linolenic acid flax. For comparison, examples of total oil content/fatty acid profile/alpha linolenic acid content of different varieties are provided in FIG. 1. Different varieties and cultivars of flax produce seeds which contain oil with different levels of ALA and different fatty add profiles. The level of ALA in mature seeds is strongly influenced by the activity of FAD3a and FAD3b genes which encode an amino acid sequence which produces a polypeptide or protein with the function of catalyzing a double bond. Furthermore, the genome of high alpha linolenic acid flax is characterized by a unique pattern of simple sequence repeat regions.

Compared to wild type Bethune (BeFAD3A.seq) sequence, the cDNA sequence of high alpha linolenic acid flax M6552 (NoFAD3A.seq) contains two deletions. The first deletion is 6 nucleotide located 40 bp from ATG. This deletion does not alter the open reading frame. The second deletion is 2 base pair deletion at 260 from the translational start site. This second deletion results in an altered reading frame and a premature stop codon at position 306. The FAD3A gene from high alpha linolenic acid flax M6552 (NoFad3A.seq) is predicted to produce a truncated and altered protein of only 100 amino acids. In comparison, the Fad3A gene from wild type Nomandy flax contains a mutation at 874 base pairs, converting an Arginine codon (CGA) to a stop codon (TGA). This wild type Normandy flax FAD3A gene is predicted to produce truncated Fad3A desaturase protein of 291 amino acids. The FAD3B gene from high alpha linolenic acid flax M6552 (NoFad3B.seq) as compared to wild type Bethune flax (BeFad3B.seq), contains 7 substitution mutations. These mutations are located at 28 (A to G), 700 (A to G), 899 1 O (A to G), 1170 (C to T), 1174 (T to C) and 1175 (G to C). These point mutations altered the amino acids: Alanine to Threonine (28), Valine to isoleucine (700), Arginine to Histidine (899), Proline to Cysteine (1174 and 1175). These substitutions will, not alter the open reading frame but are predicted to produce a FAD3b desaturase protein with altered residues. It is likely that the FAD3B protein from high alpha linolenic acid flax M6552 (NoFad3b.pro) protein still retains the enzymatic activity. Testing the biological and substrate specificities of this clone in heterologous system like yeast may provide important insights for any possible connections between this gene and unique high alpha linolenic acid oilseed flax profiles. By comparison, the wild type Normandy FAD3b desaturase gene, (NmFad3B.seq), contains a substitution mutation at 162 bp from the start site which converts Trp codon (TGG) to a stop codon (TGA). This gene is predicted to produce a truncated Fad3b protein with only 53 amino acids and is likely not functional.

### FAD3 Genes

The FAD3 gene encodes for endoplasmic omega-3/delta-15 desaturase, an enzyme responsible for the desaturation of linoleic acid (C18:2) to linolenic acid (C18:3). In Flax plants, two FAD3 genes (FAD3A and FAD3B) in particular have been reported to control linolenic content. FAD3A and FAD3B show a high degree of conservative, with about a 95% identity. In low-linolenic acid cultivars of flax, these genes have been shown to be inactive.

Compared to BeFAD3A (wt) sequence, NoFAD3A cDNA sequence contains two deletions: the first one is 6 nucleotide deletion located 40 bp from ATG, This deletion maintains the open reading frame; however, the second deletion of 2 bp length at 260 from the translational start site, results in altered and shifted reading frame and premature stop codon at 306.

The NoFad3A gene predicted to produce a truncated and altered protein of only 100 aa.

The NoFad3B gene compared to BeFad3B (wt), contains 7 substitution mutations located at 28 (A to G), 700 (A to G), 899 (A ), 1170 (C to T), 1174 (T to C) and 1175 (G to C). These point mutations altered the amino acids. Ala to Thr (28), Val to Ile(700), Arg to His (899), Pro to Cys (1174 and 1175). These substitutions do not alter the open reading frame but the point mutations change the amino acid codon for several position of the Fad3b protein. The NoFad3b protein has been demonstrated to retain the enzymatic activity SSR regions

An SSR (simple sequence repeat) is a genomic locus that contains repetitive sequence elements of typically from 2 to 7 nucleotides. Each sequence element, a repeat unit, is repeated at least once within an SSR. Examples of SSR sequences of flax include, but are not limited to: (AAT)5×, (TC)6×, (TA)8×, (TTA)5×, (GAG)6×, (TAT)5×, (TTC)6×, (CTC)5×, (TA)6×, (AT)10×.

In certain instances, the repeat unit is repeated in tandem, as shown above. In other instances, the repeat unit can be separated by intervening bases or deletions provided that at least in one instance the repeat unit is repeated in tandem once. These are referred to as “imperfect repeat,” “incomplete repeat,” and “variant repeat.”

SSR loci are preferred for determining identity because of the powerful statistical analysis that is possible with these markers. Individuals can possess different numbers of repeat units and sequence variations at an SSR locus. These differences are referred to as “alleles.” Each SSR locus often has multiple alleles. As the number of SSR loci analyzed increases, the probability that any two individuals will possess the same set of alleles becomes vanishingly small.

SSR alleles are typically categorized by the number of repeat units they contain. For example, an allele designated 12 for a particular SSR locus would have 12 repeat units. Incomplete repeat units are designated with a decimal point following the whole number, for example, 12.2.

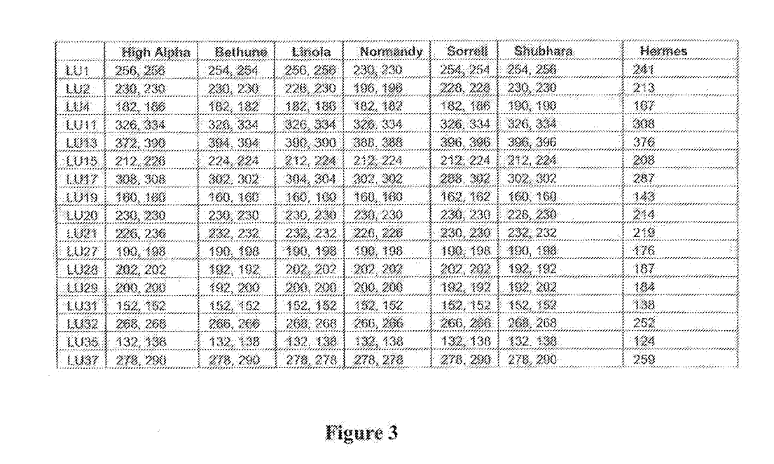

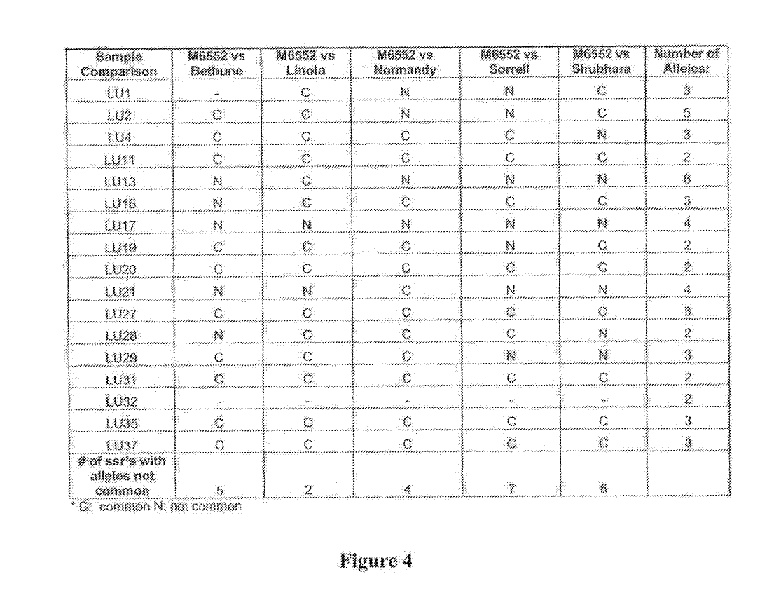

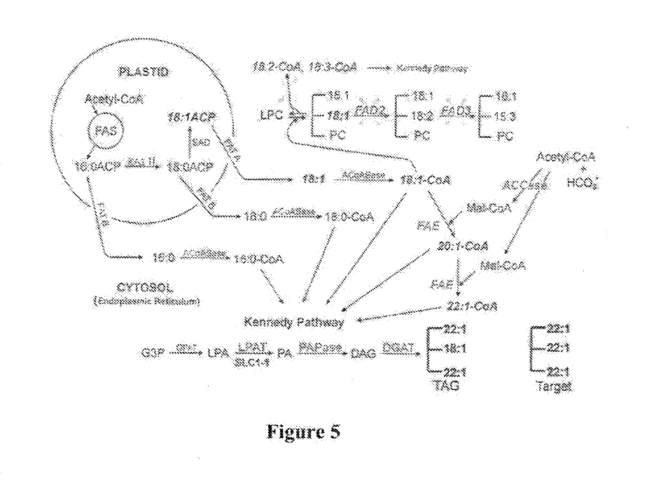

The present invention relates to simple sequence repeat region gene markers in high alpha linolenic acid flax which produce seeds containing at least 65%, 70%, or 75% omega 3 fatty acid alpha linolenic acid (C18:3). High alpha linolenic acid flax is identifiable by the characteristic and unique, simple sequence repeat regions. The loci of each SSR region tested in high alpha linolenic acid flax are associated with a unique primer sequence (FIG. 2). The length of the simple sequence repeat region at each of these loci for high alpha linolenic acid flax (high alpha), conventional wild-type flax (Bethune, Normandy, Sorrell), low linolenic acid flax (Linola), an intermediate linolenic acid flax (Shubhara) and fiber flax (Hermes) shows that each type of flax has a unique characteristic pattern of SSR region lengths (FIG. 3). A comparison of the length of the SSR region between high alpha linolenic acid flax, conventional wild-type flax (Bethune, Normandy, Sorrell), low linolenic acid flax (Linola), an intermediate linolenic acid flax (Shubhara) and fiber flax (Hermes) shows that high alpha linolenic acid flax has unique SSR regions i.e. SSR regions which are not common with other types of flax (FIG. 4). Based on SSR region data, high alpha linolenic acid flax are clearly genetically different from ‘conventional’ flax, low linolenic acid flax and fiber flax.

As will be appreciated by one of skill in the art, in some embodiments of the invention, there is provided the genome of the high alpha linolenic acid flax characterized by a unique pattern of simple sequence repeats at specified loci as shown in FIG. 3 wherein the alpha linolenic acid content of seed from said flax is 65%, 70%, or 75% or greater,

As will be appreciated by one of skill in the art, in some embodiments of the invention, there is provided a method for identifying a flax variety as high alpha linolenic acid flax by a unique pattern of simple sequence repeats at specified loci as shown in FIG. 3 wherein the alpha linolenic acid content of seed from said flax is 65%, 70%, or 75% or greater.

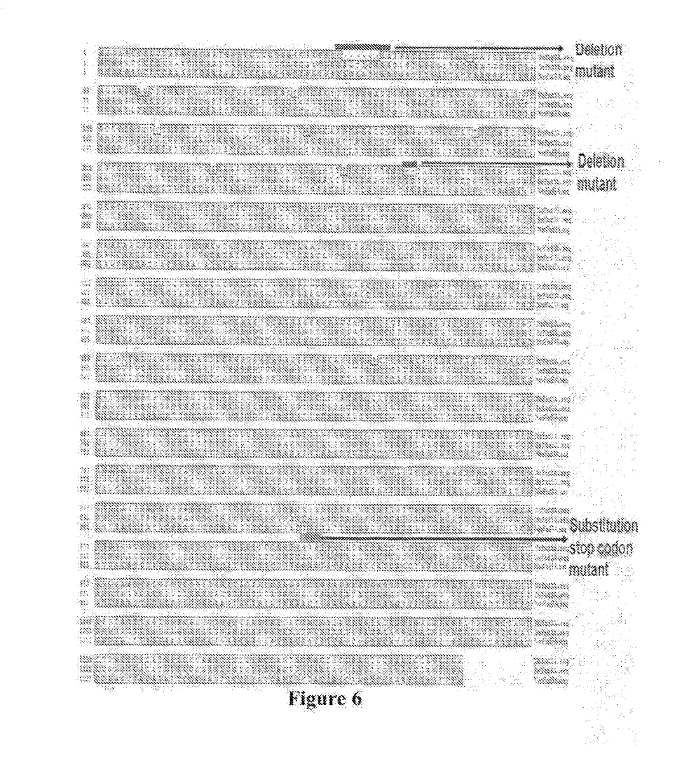

In another aspect of the invention, there is provided a purified or isolated nucleic acid molecule comprising a nucleotide sequence as set forth in FIG. 6. As will be appreciated by one of skill in the art, this nucleic acid molecule encodes the FAD3a gene isolated from high alpha linolenic acid flax wherein the FAD3a gene codes for a fatty acid desaturase used in the synthesis of alpha linolenic acid.

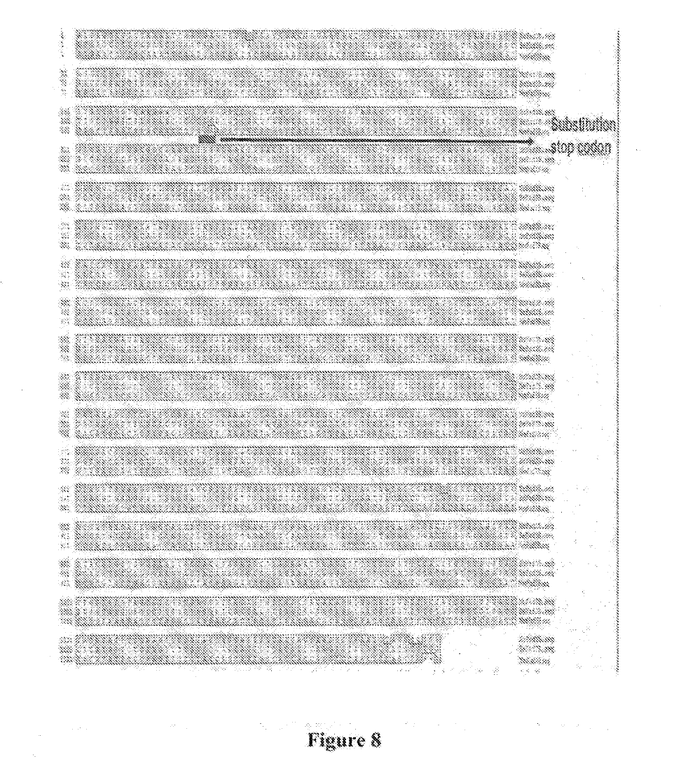

In another aspect of the invention, there is provided an isolated or purified nucleic acid molecule comprising a nucleotide sequence as set forth in FIG. 8. As will be apparent to one of skill in the art, the nucleotide sequence encodes the FAD3b gene isolated from high alpha linolenic acid flax wherein the FAD3b gene codes for a fatty acid desaturase used in the synthesis of alpha linolenic acid.

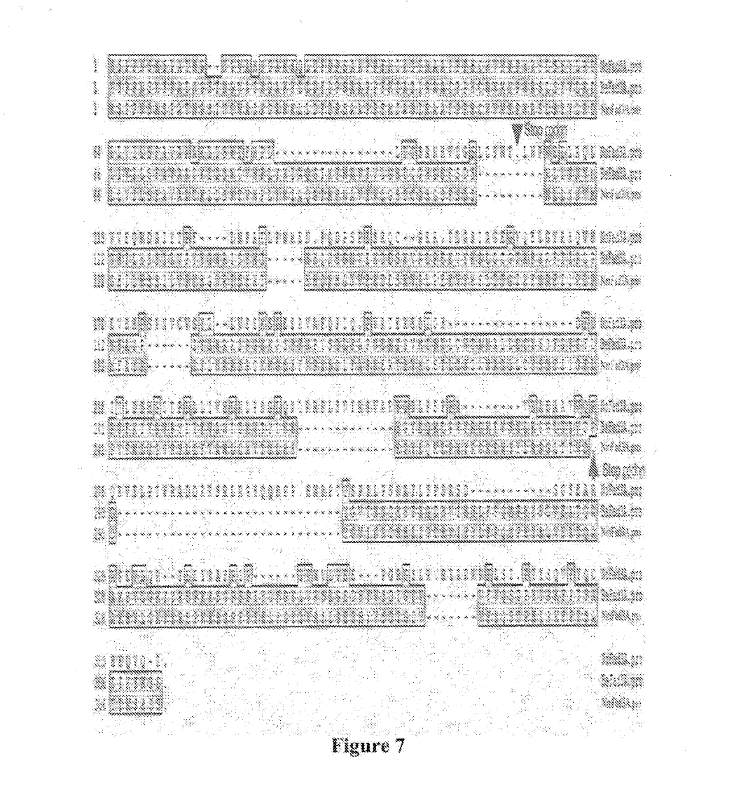

In another aspect of the invention, there is provided an isolated or purified polypeptide comprising an amino acid sequence as set forth in FIG. 7. As will be appreciated by one of skill in the art, this polypeptide is encoded by the FAD3b gene isolated from high alpha linolenic acid flax wherein the amino acid sequence produces a unique polypeptide or protein with the action of catalyzing the formation of a double bond.

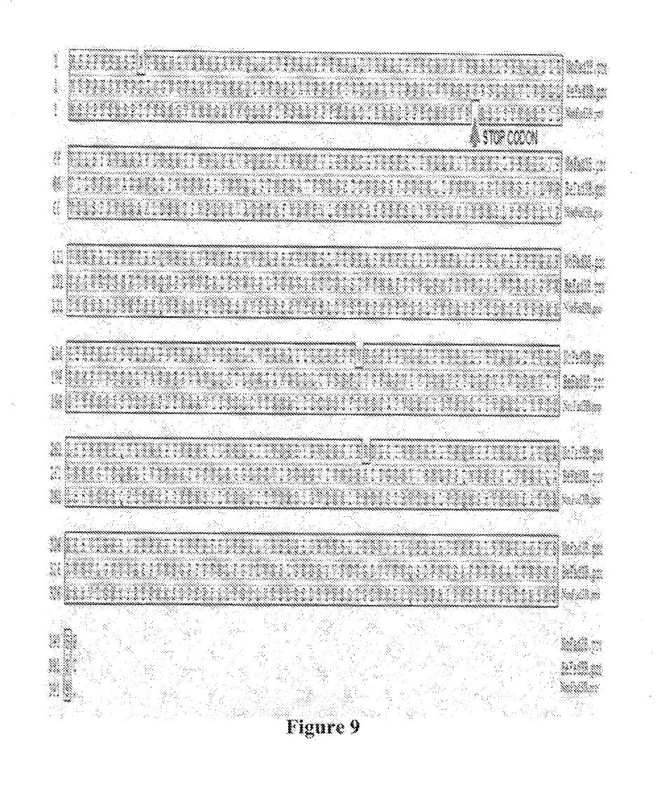

In another aspect, of the invention, there is provided an isolated or purified polypeptide comprising an amino acid sequence as set forth in FIG. 9. As will be appreciated by one of skill in the art, this polypeptide is encoded by the FAD3b gene isolated from high alpha linolenic acid flax wherein the amino acid sequence produces a unique polypeptide or protein with the action of catalyzing the formation of a double bond.

While the preferred embodiments of the invention have been described above, it will be recognized and understood that various modifications may be made therein, and the appended claims are intended to cover all such modifications which may fall within the spirit and scope of the invention.

In one embodiment, a nucleic acid molecule of the present invention comprises a nucleotide sequence which is at least (or no greater than) 50-100, 100-200, 200-300, 300-400, 400-500, 500-600, 600-700, 700-800, 800-900, 1000-1100, 1100-1181 or more nucleotides in length and hybridizes under stringent hybridization conditions to a complement of a nucleic acid molecule of: SEQ ID NO: 35 and or SEQ ID NO: 41.

### M6552 Cultivar

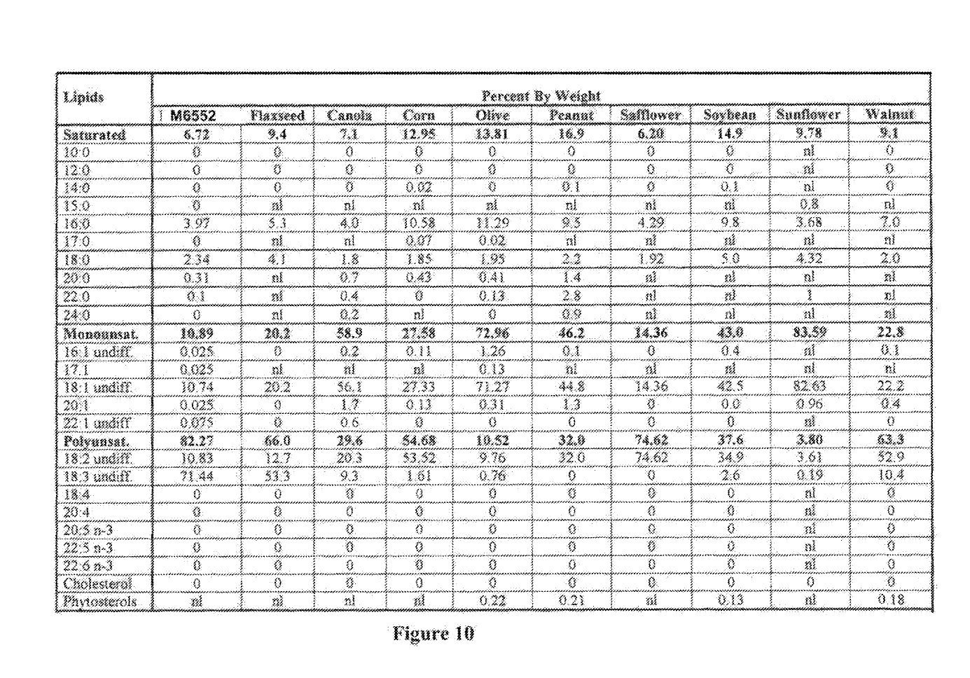

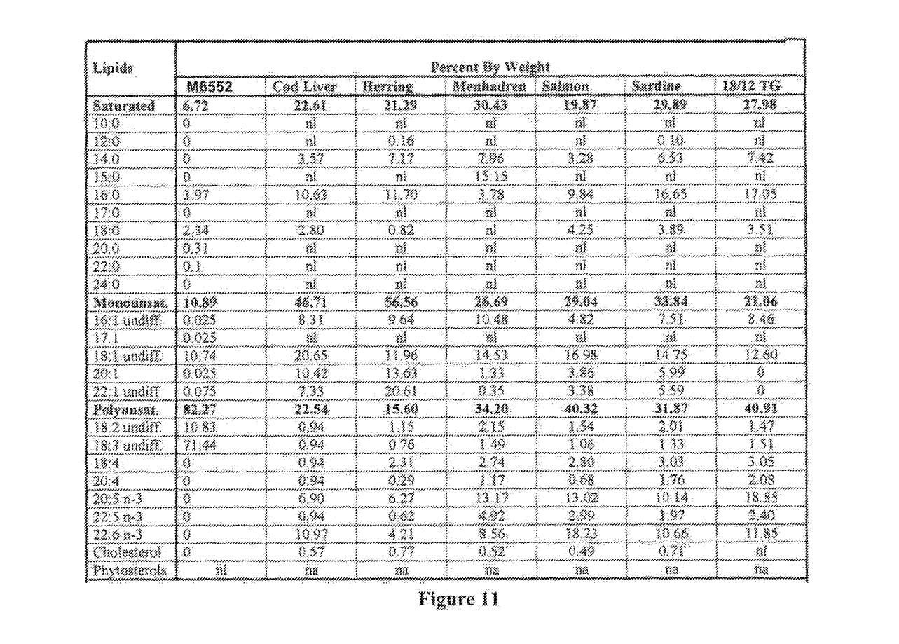

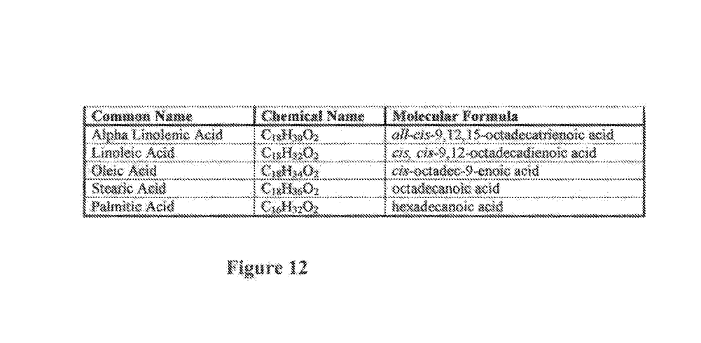

M6552 cultivar of flax was developed at the Morden Research Station, Agriculture and Agri-Food Canada, Morden, Manitoba, Canada. M6552 flaxseed oil is naturally composed of a mixture of fatty acids in the form of triacylglycerides. The fatty acid moieties in M6552 flaxseed are primarily 70±3% alpha-linolenic acid (ALA), 10±2% linoleic acid (LA), 12±2% oleic acid, 4±2% stearic acid, and 4±2% palmitic acid. Cultivar M6552 flaxseed oil is compared to conventional flaxseed oil as shown the Table of FIG. 10. It is also compared to other vegetable oils such as canola, corn, olive, peanut, safflower, soybean, sunflower and walnut oils in the Table of FIG. 11. M6552 flaxseed oil is processed and prepared as a liquid oil. M6552 flaxseed oil is a mixture of fatty acids, primarily in the form of triacylglycerides. The fatty acids are primarily alpha linolenic acid, linoleic acid, oleic acid, stearic acid and palmitic acid. Of the fatty acids present, alpha linolenic acid constitutes 68-73%, linoleic acid constitutes 9-12%, oleic acid constitutes 9 -14%, stearic acid constitutes 2-6% and palmitic acid constitutes 3-6%. Other components present in small quantities (1-2%) include sterols, tocopherols, pigments and other minor constituents. M6552 flaxseed oil is a mixture of fatty acids. The molecular formulas for alpha linolenic acid, linoleic, oleic, stearic and palmitic acid, the major fatty acid components are described herein and listed in FIG. 12.

Compared to BeFAD3A (wt) sequence, NaFAD3A cDNA sequence contains two deletions: the first one is 6 nucleotide deletion located 40 bp from ATG, it doesn't, alter the open reading frame; the second one with 2 bp deletion at 260 from the translational start site, results in altered reading frame and premature stop codon at 306.

The NoFad3A gene predicted to produce a truncated and altered protein of only 100 aa.

The NoFad3B gene compared to BeFad3B (wt), contains 7 substitution mutations located at 28 (A to G), 700 (A to G), 899 (A to G), 1170 (C to T), 1174 (T to C) and 1175 (G to C). These point mutations altered the amino acids: Ala to Thr (28), Val to Ile (700), Arg to His (899), Pro to Cys (1174 and 1175). These substitutions didn't alter the open reading frame but predicted to produce Fad3b protein with altered residues.

It is demonstrated that the NoFad3b protein still retains the enzymatic activity—it is believed the NoFAD3b contributes to unique Norcan oil profiles.

What is claimed:

1. A high alpha linolenic acid flax oil comprising a cold pressing of High alpha linolenic acid flax seed, the oil having a composition that is more than 70 wt. % alpha linolenic acid, more than 10 wt. % linoleic acid and more than 10 wt. % oleic acid.

2. The high alpha linolenic acid flax oil of claim 1 consisting of the cold pressing of High alpha linolenic acid flax seed.

3. The high alpha linolenic acid flax oil of claim 1, wherein the High alpha linolenic acid flax seed is a seed from a Linum usitatissium plant comprising a LU17 SSR of 308 bp.

4. The high alpha linolenic acid flax oil of claim 3, wherein the Linum usitatissium plant includes the whole pattern of SSRs depicted in FIG. 3 column “High Alpha”.

5. The high alpha linolenic acid flax oil of claim 1, wherein the High alpha linolenic acid flax seed is a seed from a high alpha linolenic acid flax plant having the modified genes as listed in SEQ ID NO: 35 and SEQ ID NO: 41 and expressing the amino acid sequence as listed in SEQ ID NO: 36 and SEQ ID NO: 42.

6. The high alpha linolenic acid flax oil of claim 1, wherein the High alpha linolenic acid flax seed is a seed from a high alpha linolenic acid flax plant that expresses a FAD3b protein comprising at least one of the mutations, shown in FIG. 8, of NoFad3A.pro when compared to BeFad3A.pro or NmFad3A.pro.

7. The high alpha linolenic acid flax oil of claim 6, wherein the FAD3b protein catalyzes the formation of a double bond in a linoleic fatty acid.

8. The high alpha linolenic acid flax oil of claim 1, wherein the High alpha linolenic acid flax seed is a seed from a high alpha linolenic acid flax plant that expresses a FAD3b protein comprising at least one of the mutations, shown in FIG. 9, of NoFad3.pro when compared to BeFad3B.pro or NmFad3B.pro.

9. The high alpha linolenic acid flax oil of claim 8, wherein the FAD3b protein catalyzes the formation of a double bond in a linoleic fatty acid.

10. The high alpha linolenic acid flax oil of claim 1, wherein the High alpha linolenic acid flax seed is a seed from a high alpha linolenic acid flax plant characterized by genome comprising a pattern of simple sequence repeats which has 85% equality to base pair length of the simple sequence repeats patterns of cultivar M6552 at the locus defined by primer pairs of SEQ ID NO 1 and SEQ ID NO: 2, SEQ ID NO: 3 and. SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO 22, SEQ ID NO: 23 and SEQ ID NO: 24, SEQ ID NO: 25 and SEQ ID NO: 26. SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34.

11. A High alpha linolenic acid flax seed comprising an oil having a composition that consist of at least 70 wt. % alpha linolenic acid, at least 10 wt. % linoleic acid, and at least 10 wt. % oleic acid.

12. The High alpha linolenic acid flax seed of claim 11, wherein the seed is from a Linum usitatissium plant comprising a LU17 SSR of 308 bp.

13. The High alpha linolenic acid flax seed of claim 11, wherein the Linum usitatissium plant includes the whole pattern of SSRs depicted in FIG. 3, column “High Alpha”.

14. The High alpha linolenic acid flax seed of claim 11, wherein the seed is from a high alpha linolenic acid flax plant having the modified genes as listed in SEQ ID NO: 35 and SEQ ID NO: 41 and expressing the amino acid sequence as listed in SEQ ID NO: 36 and SEQ ID NO: 42.

15. The High alpha linolenic acid flax seed of claim 11, wherein the seed is from a high alpha linolenic acid flax plant that expresses a FAD3b protein comprising at least one of the mutations, shown in FIG. 8, of NoFad3A.pro when compared to BeFad3A.pro or N m Fad3A.pro.

16. The High alpha linolenic acid flax seed of claim 15, wherein the FAD3b protein catalyzes the formation of a double bond in a linoleic fatty acid.

17. The High alpha linolenic acid flax seed of claim 11, wherein the seed is from a high alpha linolenic acid flax plant that expresses a FAD3b protein comprising at least one of the mutations, shown in FIG. 9, of NoFad3B.pro when compared to BeFad3B.pro or NmFad3B.pro,

18. The High alpha linolenic acid flax seed of claim 17, wherein the FAD3b protein catalyzes the formation of a double bond in a linoleic fatty acid.
